(19) United States
(12) Patent Application Publication
(10) Pub. No.: US 2021/0378860 A1
(43) Pub. Date: Dec. 9, 2021

(54) Arthritis Treatment System and Associated Methods

(71) Applicant: ZeoThermal Technologies, LLC, Longmont, CO (US)

(72) Inventors: David L. Basinger, Loveland, CO (US); Steven A. Schechter, Longmont, CO (US); James A. Hepp, Longmont, CO (US)

(21) Appl. No.: 17/411,298

(22) Filed: Aug. 25, 2021

Related U.S. Application Data
(63) Continuation of application No. 16/517,578, filed on Jul. 20, 2019.
Application No. 16/517,578 is a continuation-in-part of application No. 15/486,105, filed on Apr. 12, 2017, now Pat. No. 10,859,295.
(60) Provisional application No. 62/701,092, filed on Jul. 20, 2018.
(60) Provisional application No. 62/321,887, filed on Apr. 13, 2016.

(51) Int. Cl. A61F 7/02; F25B 21/02; A61F 7/08; F25B 29/00; A61F 7/00

(52) U.S. Cl. CPC A61F 7/0241; F25B 21/02; A61F 7/08; A61F 2007/0075; A61F 7/007; A61F 2007/0001; A61F 2007/0054; F25B 29/00

(57) ABSTRACT

An arthritis treatment system includes a cooling treatment mechanism for administering a cooling treatment protocol to a patient. The system also includes a heating treatment mechanism for administering a heating treatment protocol to the patient, distinctly and simultaneously with the cooling treatment protocol. The system further includes a controller for controlling both the cooling and heating treatment mechanisms. The arthritis treatment system simultaneously provides the cooling and heating treatment protocols to the patient, and the controller algorithmically adjusts the cooling and heating treatment protocols.

### PRIORITY CLAIM

This application is a continuation-in-part of U.S. patent application Ser. No. 15/486,105, filed on Apr. 12, 2017 and entitled “Cooling and Heating Platform,” which in turn claims the benefit of U.S. Provisional Patent Application No. 62/321,887, filed on Apr. 13, 2016 and entitled “Cooling and Heating Platform.” Also, this application claims the benefit of U.S. Provisional Patent Application No. 62/701,092, filed on Jul. 20, 2018 and entitled Arthritis Treatment System and Associated Methods.”

### FIELD OF THE INVENTION

The present invention relates to cooling and heating systems and, more specifically, systems and methods for simultaneous or alternating cooling and heating treatment for arthritis-related pain.

### BACKGROUND OF THE INVENTION

Cooling and heating are provided for a wide array of different end-uses. These include, but are not limited in application to, the food industry (from farming to food preparation and food service), automotive, marine, and recreational vehicles, residential and commercial heating, ventilation, and air conditioning (HVAC) systems, manufacturing and fabrication, military, and medical applications. Most cooling and heating systems involve heat transfer. That is, either heat is added or removed to provide the desired heating or cooling respectively.

In particular, arthritis is a common condition, which affects an estimated 91 million Americans (“Arthritis by the Numbers—Book of Trusted Facts & Figures,” Arthritis Foundation, 2018). There are nearly 100 different types of arthritis, all resulting in some form of inflammation and pain for the sufferer. As an example, osteoarthritis is a particular type of arthritis, which is commonly treated with pain medication, cold and hot wraps, and exercise. Hot or warm compresses can be used to help decrease pain and joint stiffness by increasing blood flow, and thus lubricating fluids such as lymphatic fluids around the affected joints. However, when the joints become inflamed, such as can occur when white blood cells are produced in reaction to a trigger and the blood cells concentrate around the joints of the arthritis patient, the tissues surrounding the joints can become swollen and physically hot. While the swelling can be reduced with the application of cold compresses, arthritis sufferers are often highly sensitive to cold temperatures and cannot tolerate the level of cold required to reduce the swelling.

### SUMMARY OF THE INVENTION

In accordance with the embodiments described herein, an arthritis treatment system includes a cooling treatment mechanism for administering a cooling treatment protocol to a patient. The system also includes a heating treatment mechanism for administering a heating treatment protocol to the patient, distinctly and simultaneously with the cooling treatment protocol. The system further includes a controller for controlling both the cooling and heating treatment mechanisms. The arthritis treatment system simultaneously provides cooling and heating treatment protocols to the patient, and the controller algorithmically adjusts the cooling and heating treatment protocols.

In accordance with another embodiment, the controller includes a user input interface for collecting user input from the patient, and the controller is configured for taking into account the user input for algorithmically adjusting the cooling and heating treatment protocols accordingly. The user input interface is also configured for collecting patient information including at least one of patient weight, patient height, patient age, body temperature, patient sex, and diagnosed ailment.

In still another embodiment, the system includes a controller configured for tracking patient usage data, including system settings during each treatment session. Further, the controller is configured for adjusting the cooling and heating treatment protocols in accordance with the patient usage data.

In yet another embodiment, the system includes sensors for monitoring patient vital signs, including temperature, pulse rate, and oxygen saturation.

### BRIEF DESCRIPTION OF THE DRAWINGS

FIG. 1 is a diagrammatic overview of an example cooling and heating platform.

FIG. 2 illustrates an example application configuration of the cooling and heating platform.

FIG. 3 is an illustration of an arthritis treatment system, in accordance with an embodiment.

FIG. 4 is a process flow chart illustrating an exemplary method for using the arthritis treatment system, in accordance with an embodiment.

FIG. 5 is a graph representing an exemplary use of the arthritis treatment system by a user, in accordance with an embodiment.

FIG. 6 is a numerical representation of the temperatures plotted in the graph of FIG. 5.

FIG. 7 is a graph showing the temperature setting trend by the exemplary user.

FIG. 8 is a graph showing exemplary heating and cooling treatment profiles, illustrating the target temperatures and actual temperatures, as measured in the hot and cold liquid reservoirs, as a function of time.

### DETAILED DESCRIPTION OF EMBODIMENTS OF THE INVENTION

A cooling and heating platform is disclosed. In an example, the cooling and heating platform may be implemented as a cooling and heating platform that is inherently operating at a selected temperature, controlled via vacuum, hygroscopic, electrostatic system(s), and/or a heating element, e.g., in a combinatory manner. The cooling and heating platform may be implemented in a wide variety of cooling, refrigeration, and/or heating applications.

In an example, the cooling and heating platform manages pressure within an operating chamber to maintain a steady operating temperature based on the boiling point of an “operating fluid.” In an example, the operating liquid is an inexpensive and environmentally friendly “refrigerant.”

By way of illustration, the refrigerant may be water-based and thus ecologically-friendly. An example water-based refrigerant includes, but is not limited to, distilled water. However, other operating liquids may also be implemented. Configurations utilizing a variety of other operating liquids can operate in different temperature ranges, allowing for heating and chilling solutions for an expanded range of applications.

Unlike standard refrigeration or ice, the example cooling and heating platform provides chilling to a specific temperature. The cooling and heating platform is not limited to extreme chilling that requires external control to achieve the desired temperature. This is a particularly important aspect in applications such as, but not limited to, physical therapy. In physical therapy, using too cold of a temperature (e.g., freezing) can have adverse health effects.

The cooling and heating platform is a viable replacement for many chilling/refrigeration devices that are based on the use of standard refrigerants (e.g., chlorofluorocarbons (CFCs) and their replacements). As such, cooling technologies based on the cooling platform may be implemented to reduce the climate impacts from world-wide use of CFCs and their replacements.

Before continuing, it is noted that, as used herein, the terms “includes” and “including” mean, but is not limited to, “includes” or “including” and “includes at least” or “including at least.” The term “based on” means “based on” and “based at least in part on.”

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity. Like numbers refer to like elements throughout.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items and may be abbreviated as “/”.

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adjacent to” another element or layer, there are no intervening elements or layers present. Likewise, when light is received or provided “from” one element, it can be received or provided directly from that element or from an intervening element. On the other hand, when light is received or provided “directly from” one element, there are no intervening elements present.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

In particular, the term “operating liquid” means any suitable matter to absorb energy via change of phase. The term “operating chamber” means any suitable partially or fully sealed vessel or container that houses a phase-change mechanism.

The term “heat exchanger” means a device used to transfer heat from one medium to another.

The term “application interface” means any mechanism that enables the transfer of thermal energy between the cooling/heating platform and an application that utilizes the heating/cooling provided by the platform. This may include, but is not limited to, an “application fluid” that physically transfers heat by flowing or circulating through a heat exchanger and the application.

In addition, the term “thermal battery” as used herein means any suitable device or matter to store thermal energy. A thermal battery, e.g., additional operating liquid, provides the ability to satisfy burst chilling/heating requirements that exceed the instantaneous capacity of the device.

The term “operating liquid supply” means a device that adds operating liquid to the operating chamber.

The term “hygroscopic material” means a material that adsorbs operating liquid vapor from the platform, e.g., from the operating chamber.

The term “electrostatic device” means a device that causes operating liquid vapor atoms/molecules to move in a desired path due to electrostatic fields, e.g., attracting ionized vapor to an anode or cathode for removal from the operating chamber.

The term “bypass switch” means a device that reroutes application fluid depending on the mode selected by the user.

The term “control system” means a system that monitors performance, maintains, displays, and/or records the state, and controls the platform relative to a desired mode selected by the user.

The term “overpressure” means pressure above ambient atmospheric pressure.

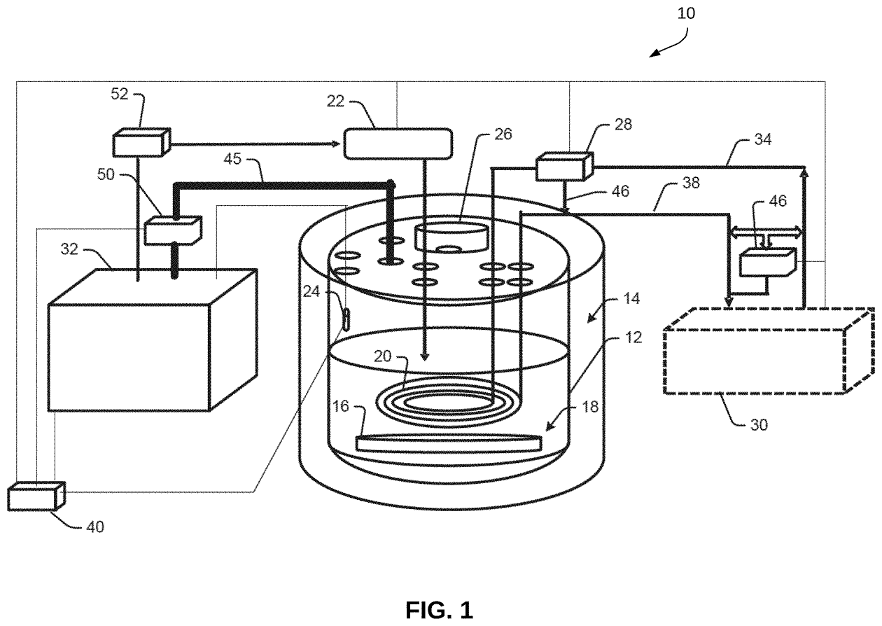

FIG. 1 is a diagrammatic overview of an example cooling and heating platform 10. The example cooling and heating platform 10 includes a thermally isolated operating chamber 12. A thermal isolation layer 14 is provided around the operating chamber 12 and a heat exchanger 20. The operating chamber 12 includes a thermal battery 16 and an operating liquid 18.

The example cooling and heating platform 10 also includes an operating liquid supply 22. Example configurations of the cooling and heating platform 10 may include a total load of operating liquid 18, e.g., to sustain operation through a nominal operational period.

The operating liquid supply 22 may include a mechanism to reload and restart the device (e.g., open, refill, and then reestablish vacuum).

In another example, operating liquid 18 can be added during operation by introducing operating liquid 18 from the operating liquid supply 22 (e.g., an external source) directly into the operating chamber 12 without breaking vacuum.

Example implementations may include at least one sensor 24, e.g., temperature, pressure, operating liquid level, on the interior of the operating chamber 12. A vapor removal mechanism 26 may be provided. A fluid circulating pump 28 may be provided to move the operating liquid 18 between the operating chamber 12 and an application 30. The pump 28 may also be implemented to move the application fluid through the heat exchanger 20.

The cooling and heating platform 10 may be configured with one or more connectors that provide access to heat exchanger 20. The connectors may be commercially available (e.g., standard water hose connection), or specifically designed to a particular application. A pressure management device 32, e.g., a vacuum pump, and an operating liquid recovery mechanism 52 may be provided. Control connections may be provided to control the pressure management device and operating liquid recovery mechanism 52 based on feedback from at least one sensor 24 for the operating chamber 12 and/or for the application 30 to a control system 40 to orchestrate any/all elements of the platform.

The cooling and heating platform 10 can be incorporated into any application 30 that utilizes traditional chilling/refrigeration and can also be configured to support a wide range of cooling and heating applications. The cooling and heating platform also supports many, if not most, everyday chilling/refrigeration applications 30 and a range of cooling and/or heating applications 30. Examples of applications 30 include, but are not limited to, an in-line fluid cooler/heater and a portable cold storage device.

An in-line fluid cooler/heater may have application to the following:
a. Liquor brewing (beer, whiskey, etc.)—brewers struggle with cooling wort fast enough so as to mitigate wort loss and contamination.
b. Dairy farming—when cooling milk recovered during the dairy milking process, massive quantities of water are used to cool milk during delivery from collection to processing by pipes on the farm. The device eliminates all water waste by cooling collected milk before receipt by processing.
c. Breast milk processing—when breast milk is pumped, it mused be cooled before refrigeration is allowed; current process takes longer than desired which risks contamination and loss. The device cools breast milk from body temperature to 40° F., ready for storage.
d. Food service (microbreweries, brew-pubs, restaurants)—Brew masters struggle with ways to improve the quality of the consumer's beer experience. Serving beer at the optimum temperature for taste is desirable but difficult. The device allows beer to be served at its intended or optimum temperature. In addition, in the fight for market share, breweries compete for a tap presence in restaurants, taverns, bars, etc. Other foods may require warming.

A portable cold storage device may have application to the outdoor recreation (boating, RV, hunting, camping, etc.) industry—Consumers want convenience and good products to enjoy their outdoor activities. During recreational activities, people are always running for more ice. Current built-in boat coolers only hold ice for a few hours. With the cooling system retrofitted into an existing built-in cooler or incorporated into new cooler designs, purchasing a premium cooler will no longer be necessary.

A vacuum-based version as detailed above may have application to the following:
a. Commercial construction.
b. Residential construction.
c. Automotive (cars and RVs).

A version for manufacturing-based industries may have application to the following (e.g., for equipment and process cooling):
a. Plastics.
b. Foundries.
c. Printing.
d. Rubber.
e. Plating.
f. Machine Fabrication.

A food service version may have application to the following:
a. Residential refrigerators.
b. Food service walk-in coolers (restaurants, etc.).
c. Food retailers (grocery stores, wholesalers, liquor stores, etc.).

A medical or therapy-based version may have application to the medical (in-patient/out-patient, sports/physical therapy, etc.)—since the main premise in medicine is all about healing, the medical industry actively seeks faster recovery times in order to improve healing success rates. The device provides hot and cold therapy at therapeutic temperatures within specific limits determined to be medically safe.

A transportation-based version may have application to the following:
a. Medical (organ transport—ground or air).
b. Food (food transport—ground or air).
   Example configurations of the cooling and heating platform 10 may be provided for different operating temperatures to support other chilling and/or heating applications. The operating liquid 18 may be selected based on design considerations such as, but not limited to, optimizing the ability to maintain the target operating temperature required for the application. Other considerations may include, but are not limited to, the pressure/vacuum and environmental/safety considerations of the operating liquid 18.

In an example, the cooling and heating platform 10 may be portable (e.g., hand-carried), semi-portable (e.g., movable with the assistance of a hand truck, or similar), or fixed (e.g., requiring heavy equipment to be moved.).

Example operation of the cooling and heating platform 10 is based on maintaining the pressure in a chamber or other vessel 12 containing the operating liquid 18 at a level of vacuum/overpressure (e.g., from pressure management device 32 and operating liquid recovery mechanism 52) such that the boiling point of the operating liquid 18 corresponds to the target chilling (or heating) temperature of the device or application 30. Chilling/refrigeration is provided by passing an application fluid to be chilled or heated (e.g., within return line 32) through a heat exchanger 20 (e.g., coils) immersed in the operating liquid 18 within the operating chamber 12 and to the application 30 (e.g., via supply line 38).

For the chilling configuration, having water as the operating liquid 18 in the operating chamber 12, the level of vacuum may be maintained by mechanical pumping and/or, for example, the use of hygroscopic materials such as, but not limited these two, or similar mechanisms that remove water vapor from the operating chamber 12.

The chilling capacity of the cooling and heating platform 10 is determined primarily by the heat exchanger implementation and the capacity of the cooling and heating platform 10 for removing operating liquid vapor from the operating chamber 12. The platform may be configured to maintain the operating liquid in its liquid state in order to maximize the mixing effect of boiling, but configurations cause the operating liquid to change state to solid are also possible. Phase change of the subsequent solid form of the operating liquid back to liquid form (melting) and/or vapor (sublimation) may be incorporated into the operation of the platform.

For applications that require higher chilling capacities in bursts, the device may include a thermal battery 16 of additional operating liquid and/or other material(s) with suitable heat capacity that increases the heat capacity of the operating chamber 12 to the level desired to support the thermal load from burst chilling/heating. The normal chilling/heating function of the operating chamber 12 recharges the thermal battery 16 between bursts. The thermal battery may be located within the operating chamber 12 or externally.

The overall device behavior can be controlled with device control system 40 based on inputs from the device or application including, but not limited to, temperature, pressure, flow, and/or other sensors. The device control system 40 can operate attached devices, e.g., pressure management device 32, bypass switch 46, circulating pump 28, and operating liquid supply 22.

Operating chamber 12 is connected to pressure management device 32 through vacuum line 45.

For configurations where the operating chamber 12 is providing cooling, the heating bypass mechanism 46 can direct the application fluid to bypass the operating chamber 12 and pass through a heating element either integrated or external to heating bypass mechanism 45. This permits a single device to support heating and cooling applications separately or cyclically when alternating heating/cooling cycles are desired.

Before continuing, it should be noted that the examples described above for FIG. 1 are for purposes of illustration and are not intended to be limiting. Other devices and/or device configurations may be utilized to carry out the operations described herein.

The example configuration of the cooling and heating platform 10 shown in FIG. 1 includes a thermally-isolated operating chamber 12. A thermal isolation layer 14 is provided around the operating chamber 12. The operating chamber 12 includes a thermal battery 16, an operating liquid 18, and a heat exchanger 20. The example cooling and heating platform 10 also includes a pressure management device 32 and an operating liquid supply 22.

In addition, the example cooling and heating platform 10 shown in FIG. 1 includes a vapor recovery system 50. The vapor recovery system 50 removes operating liquid 18 from vapor formed in the operating chamber 12 via operating liquid recovery mechanism 52. The operating liquid recovery mechanism 52 may include a mechanism to recycle operating liquid 18 by condensing the removed vapor (e.g., including any baked out of the hygroscopic material). The vapor recovery system 50 also returns the operating liquid 18 to an operating liquid supply 22 for return to the operating chamber 12.

In an example, the vapor removal system 50 includes hygroscopic material for removal of water vapor. Another example is where a vapor removal mechanism utilizes an electrostatic approach, similar to removing particulates from power plant and other exhausts (e.g., where the operating liquid 18 is not water-based).

Various configurations of the cooling and heating platform may permit recharging, reloading, and/or replacing vapor removal material in the vapor removal mechanism 50. The vapor removal material may include hygroscopic materials or their equivalent in non-water-based configurations. An example vapor removal mechanism 50 may include the mechanical replacement of a “cartridge” containing the vapor removal material. Another example vapor removal mechanism 50 may include a mechanism to add additional fresh material to the liquid recovery system 52. An example vapor removal mechanism 50 may also include mechanism that seals a cartridge or other container of the liquid recovery system 52 from the operating chamber 12. The vapor removal material may be exposed to the atmosphere and then dried (e.g., via a heater, or some other method that is tailored to the specific material used in the configuration).

The operations shown and described herein are provided to illustrate example implementations. It is noted that the operations are not limited to the ordering shown. Still other operations may also be implemented.

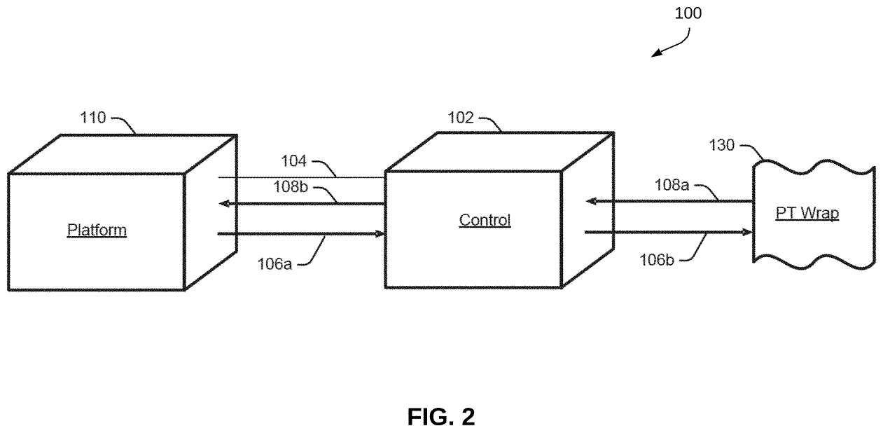

FIG. 2 is diagram 100, illustrating an application configuration of the example cooling and heating platform 9 e.g., shown in FIG. 1). In this example, the cooling and heating platform is implemented as a cycling chiller/heater platform 110 and can be applied to a physical therapy application 130.

In an example, the physical therapy application 130 may include a therapy wrap (e.g., to be placed on a body, such as an ankle wrap). The cycling chiller/heater platform 110 may be operatively associated with a controller 102 for the therapy wrap. The controller may include control electronics and/or software to implement a thermal control and circulating pump.

The cycling chiller/heater platform 110 may receive feedback 104 from the controller 102. The feedback can be utilized to control temperature to the therapy application 130. Fluid output lines 106a-b deliver the temperature-controlled application fluid to the physical therapy application 130 (e.g., the ankle wrap). Fluid return or input lines 108a-b return the application fluid to the chiller platform 110 to maintain the desired temperature.

Of course, the example shown and described with reference to FIG. 2 is only illustrative of an example implementation of the cooling and heating platform disclosed herein. Still other applications 130 are contemplated as being within the scope of this disclosure, whether specifically called out or note, as will be readily understood by those having ordinary skill in the art after becoming familiar with the teachings herein.

It is noted that the examples shown and described are provided for purposes of illustration and are not intended to be limiting. Still other examples are also contemplated.

The cooling and heating platform described above can be adapted specifically for treatment of arthritis by allowing the gradual reduction of the cooling side temperature, thus only applying as much cold as the patient can tolerate at any particular time. That is, rather than placing an ice pack at 32° F. on the arthritis patient, who will likely not be able to handle such a cold temperature and will immediately give up on the treatment before therapeutic effects can take place, the arthritis treatment system described herein will start at a cooling temperature that the patient can tolerate (e.g., even 70° F. or higher). Then, the system allows either the patient or an automated process to gradually reduce the temperature as his/her tolerance to cold is increased. That is, the arthritis treatment system described herein recognizes that the variability in an individual arthritis patient's ability to tolerate cold temperatures. Optionally, the arthritis treatment system tracks the patient's use of the system and encourages the patient to keep the cold on for increasingly longer periods of time such that the patient becomes conditioned to the cold temperatures and can therefore tolerate cold therapy for longer periods of time.

While the initial temperature in the 70° F. range may not be immediately therapeutic, the intent of the arthritis treatment system described herein is to help the patient to gradually become accustomed to colder temperatures, thus eventually reaching the beneficial therapy temperatures in the 46° F. to 65° F. range, for example. While a range of temperatures may be helpful for treatment of arthritis-related inflammation, there is agreement among medical professionals that cold therapy temperatures of 50° F. to 59° F. is optimal for longer term treatment (see, for example, “Cryotherapy: A Review of the Literature” (http://www.chiroaccess.com/Articles/Cryotherapy-A-Review-of-the-Literature.aspx?id=0000070, accessed Mar. 22, 2018); MacAuley DC, “Ice Therapy: How Good Is the Evidence,” Int J Sports Med 2001, 22, 379-384; MacAuley DC, “Do Textbooks Agree on Their Advice on Ice?” Clin J Sports Med 2001, 11, 67-72; Bleakley C, et al., “The Use of Ice in the Treatment of Acute Soft-Tissue Injury,” The Am J Sports Med 2004, 32(1), 251-261).

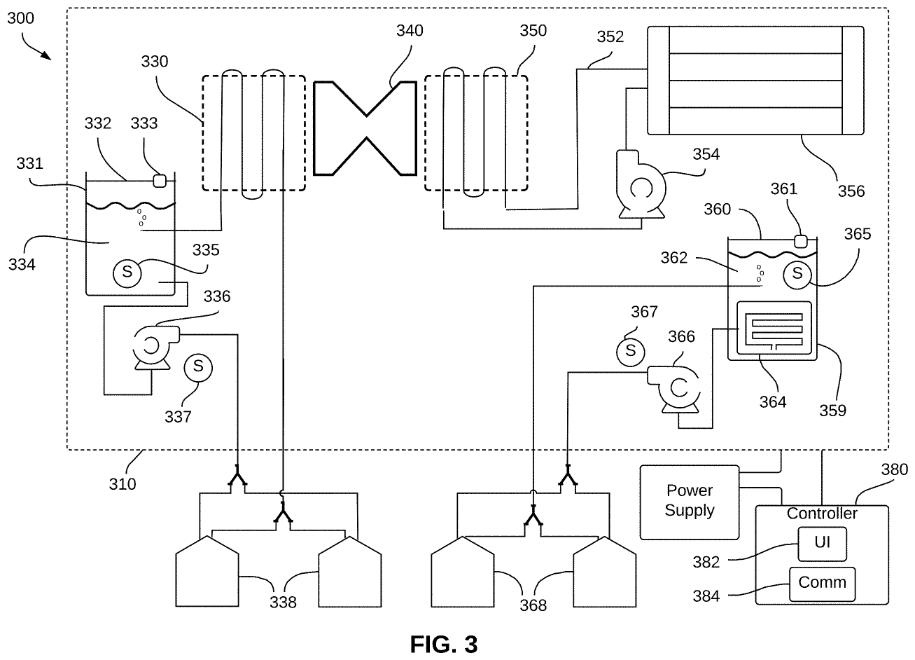

A schematic representation of an exemplary embodiment of an arthritis treatment system is shown in FIG. 3. An arthritis treatment system 300 includes a cooling/heating mechanism 310, which is controlled by a controller 380 and interfaced with a user via multiple pads. A power supply 312 supplies power to both cooling/heating mechanism 310 and controller 380, although alternatively cooling/heating mechanism 310 and controller 380 can each have its own power supply. Cooling/heating mechanism 310 includes a cold block 330 connected with a cold tank 331, which is covered by a lid 332 optionally including a pressure release valve 333. Cold tank 331 holds a cooling liquid 334. Cooling liquid 334, the temperature of which is optionally monitored by a sensor 335, is pumped by a first pump 336 into one or more cold pads 338. The temperature of cooling liquid 334, upon exiting from pump 336, can optionally be monitored by a sensor 337. Cooling liquid 334 travels through cold pads 338 back into cold block 330, which again reduces the temperature of cooling liquid 334 to a desired temperature. Cold block 330 is connected via a Peltier cooler 340 to a hot block 350, which includes a heat transfer liquid contained in tubing 352. The heat transfer liquid is pumped by a second pump 354 through a radiator 356, at which the temperature of the heat transfer liquid is dissipated.

For instance, the temperature of the cold liquid can be adjusted in a number of different ways:
By turning Peltier cooler 340 off, the patient body heat and the heat of the surrounding air will slowly transfer to cold pads 338 and cooling liquid 334, thereby, heating the pad.
By changing the polarity of the voltage to Peltier cooler 340, Peltier cooler 340 in thermal contact with the fluid will begin to warm rather than cool. That is, switching the polarity of Peltier cooler 340 causes the heating side and cooling side to switch thus increasing the temperature of cooling liquid 334 circulating through cold pads 338.
By placing an additional heating element within the cold reservoir in order to heat the liquid within the cold reservoir as desired.
Slowing the average flow rate of pump 354 that runs from the hot side of Peltier cooler 340 to radiator 356, will cause the hot side and cold side of Peltier cooler 340 to warm up, thus raising the temperature at cold pads 338.
Slowing the average fan speed, blowing through radiator 356, will also cause both sides of Peltier cooler 340 to warm, thus increasing the temperature of cold pads 338.
   Above methods for adjusting the temperature at cold pads 338 affect the temperature with different rates of change. Combinations can be used to obtain the rate of temperature change required by the particular circumstances. In an example, the fluid in the system may be a mixture of non-toxic and non-conductive propylene glycol mixed with de-ionized water.

Continuing to refer to FIG. 3, cooling/heating mechanism 310 further includes a heat tank 359, which is covered by a lid 360 optionally including a pressure release valve 361. Heat tank 359 holds a heating liquid 362. Heating liquid 362, the temperature of which is monitored by a sensor 365, is heated to a desired temperature by a heating element 364 located within heat tank 360. A third pump 366 pumps heating liquid 362 into one or more hot pads 368. The temperature of heating liquid 362, upon exiting from pump 366, is optionally monitored by a sensor 367. Heating liquid 362 then returns to heat tank 360 for reheating. Finally, a controller 380 controls the settings and status of the various components of system 300, such as sensors 335, 337, 365, and 367, as well as Peltier 340, radiator 356, first pump 336, second pump 354, third pump 366, heating element 364, as well as a user interface 382 and an optional, communication module 384. User interface 382 can include, for example, a touch screen or other mechanism for receiving patient input, as well as for issuing visual and aural instructions (e.g., via screen prompts and/or recorded messages). Communication module 384 can include, for example, Bluetooth, wireless, and/or cellular communication mechanisms for communicating with another device or the cloud, thus receiving instructions (e.g., specific treatment protocols prescribed by a physician, or system updates) and transmitting data (e.g., system registration with user profile information prior to initial use, usage data and patient feedback). Controller 380 can also include memory and one or more processors for regulating the treatment system and aggregating usage data, for instance, treatment parameters, patient information (e.g., body temperature, blood pressure, type of ailment, current status of ailment, age, sex, height, weight, current mood, general health) as well as timing of missed sessions. Such information can be collected by voice input or text input via the user interface. When anonymized and secured for compliance with the Health Insurance Portability and Accountability Act of 1996 (HIPAA), such information from multiple users can be aggregated for analytical purposes,

It is noted that vacuum sealing of the cooling and heating circuits is not necessary for the operation of arthritis treatment system 300, with the appropriate selection of pumps and liquid levels. Optionally, pressure sensors monitor the pressure in one or more of the fluid lines to sense if there is a sudden loss of pressure, which would indicate a leak. Additionally, by monitoring the pump's current flow or pressure changes, the control system can sense when there is a leak or when the pad is released from the patient. FIG. 3 shows the symbol “S” to denote placement of temperature and/or pressure and flow sensors as desired.

In an embodiment, the cold surface of Peltier cooler 340 can be insulated from the hot surface of Peltier cooler 340 in order to improve the performance of both sides of Peltier cooler 340. While the cold and hot surfaces of Peltier cooler 340 are generally in close proximity, especially in the less expensive Peltier devices, these surfaces can also be extended through heat sinks, which can add to the cost of the overall apparatus. In order to improve performance while keeping the cost and weight low, the cold side of Peltier cooler 340 can be insulated from the hot side using a lightweight insulation material, such as Styrofoam or aerogel. For example, by covering cold block 330 and Peltier cooler 340 surface, including possibly around the edges and sealing the edges of the insulation with an appropriate adhesive, the cooling performance of Peltier cooler 340 can be improved over a version of the device without the additional insulation.

The overall dimensions of arthritis treatment system 300 can be reduced for portability. For example, a prototype system measures 15-inches by 15-inches by 9-inches, and further reduction to approximately 10-inches by 10-inches by 6-inches or less, mainly limited by the size requirements for the Peltier and radiator. Alternative cooling mechanisms can be used for further reduction in size, without deviating from the spirit of the present disclosure.

In some cases, arthritic patients have difficulty applying and adjusting the pad for the best fit to their affected joint because they may not have full use of their limbs. This physical limitation is further exacerbated by heavy or stiff tubing to pad. Furthermore, adding insulation the tube also creates additional stiffness and weight. To reduce these component limitations, the tubing insulation within approximately 2 feet from the pad can be eliminated, thus reducing the weight of the tubing, for example. Alternatively, a very thin and flexible insulation, such as aerogel and similar materials, can be used instead of conventional insulation materials. Tubing should be chosen such that they are flexible and lightweight. For example, silicon with low thermal conductivity properties is a good material choice for tubing. There are a variety of factors to consider in the tube selection including, but not limited to, flexibility, weight, thermal performance, durability, and fluid pressure and flow requirements.

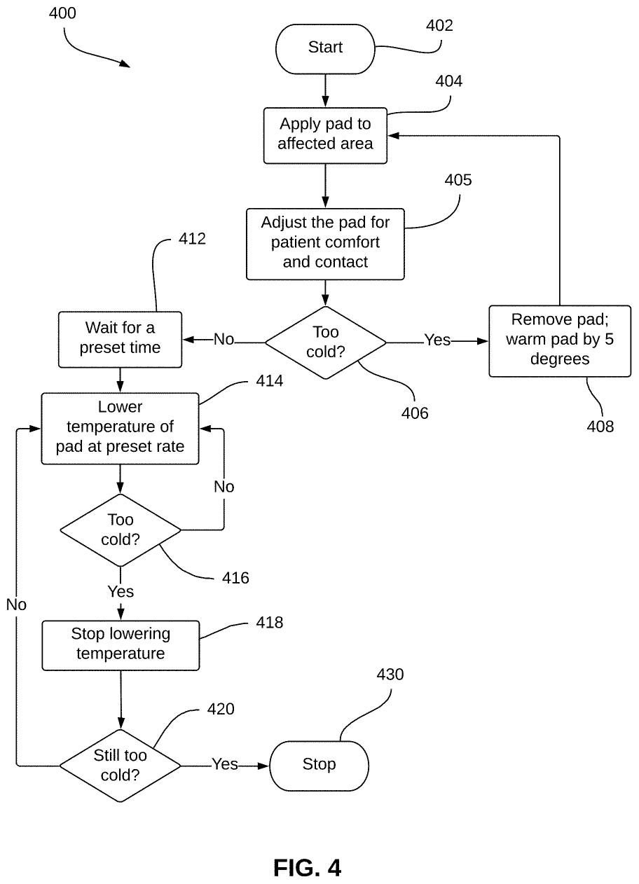

Referring now to FIG. 4, a process 400 is shown in a flow chart illustrating an exemplary process of using arthritis treatment system 300 is shown, in accordance with an embodiment. This process is used to gauge a user's tolerance for cold temperatures at the start of a new treatment. Assuming the cooling side temperature is at room temperature, and this is the first time the patient is using the system, process 400 begins with a start step 402, where the system is initialized. The cold pad (such as cold pad 338 of FIG. 3 is gently applied to an affected area on the patient in a step 404. If multiple pads are connected to the system, then multiple pads can be simultaneously applied to different areas of the patient. For example, one or more cold pads can be applied to inflamed joints, while one or more heating pads can be applied to stiff yet not inflamed joints. In a step 405, the pad is adjusted for patient comfort and optimum contact with the treatment area until the patient is satisfied with pad-to-affected-area contact and fastening comfort.

Still referring to FIG. 4, a decision 406 is made by the user whether the cold pad temperature is too cold. If the answer to decision 406 is YES, then the pad is removed from the patient and warmed by a certain amount, such as by 5° F. or some other value as displayed on the control system display, in a step 408. The warming of the pad can be accomplished by, for example, one of the methods described above. If the cold pad temperature is tolerable and the answer to decision 406 is NO then, the pad is kept on the patient for a preset time period (e.g., 3 minutes) in a step 412. Following the preset time period, the cold pad temperature is automatically reduced by a preset rate (e.g., 1° F. per minute) in step a 414. After each incremental lowering of the cold pad temperature, the patient has the option to indicate whether the temperature is too cold or is tolerable in a decision 416. If the temperature is tolerable, then process 400 returns to step 414 to continue reducing the cold pad temperature. If the perceived temperature by the patient is too cold, then the temperature of the cold pad is kept at the same level in a step 418. In a decision 420, the patient determines whether the cold pad temperature is still too cold. If the answer to decision 420 is NO, then process 400 returns to step 414 to continue to reduce the cold pad temperature by preset increments. If the answer to decision 420 is YES the cold pad temperature is too cold, then process 400 is ended in a stop step 430. In this way, the patient controls the temperature of the cold pads, while still pushing the edge of tolerance. This test method results in a temperature value that is too cold for the user by 1° F. For example, the “too cold” temperature value is 67° F., then the baseline value is set to 68° F. for that particular treatment session. Algorithms can then be used to attempt to lower this value gradually over multiple sessions until the “too cold” temperature is within the range of therapeutic temperatures (50° F.-59° F.)

The arthritis treatment system can optionally track the patient usage data and use the collected data to customize the treatment experience to the patient by modifying the temperature reduction scheme. The patient can be instructed by his/her medical provider to start at a tolerable temperature (e.g., 5 degrees above the “too cold” temperature found above), then reduce the temperature of the treatment session gradually to or below the baseline found above.

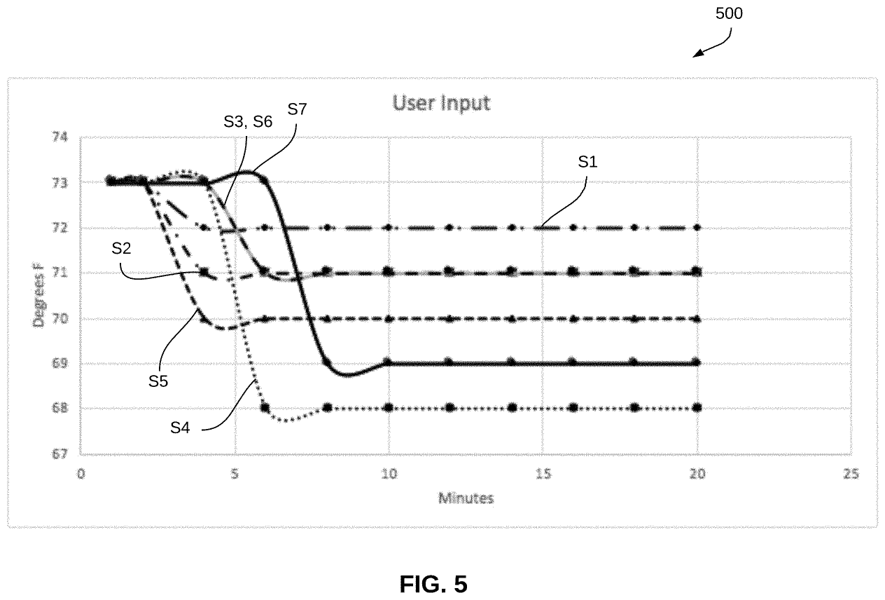

FIG. 5 shows a chart 500 showing an exemplary data set collected for a patient starting treatment at 73° F., then incrementally reducing the cold pad temperature over time and multiple uses. In the example shown in FIG. 5, the patient underwent six 20-minute treatment sessions with various temperature settings. It can be seen that, for this particular patient, he/she kept the temperature setting at the same level for the first two minutes of the treatment session. Also, the lowest temperature tolerated by the patient was 68° F.

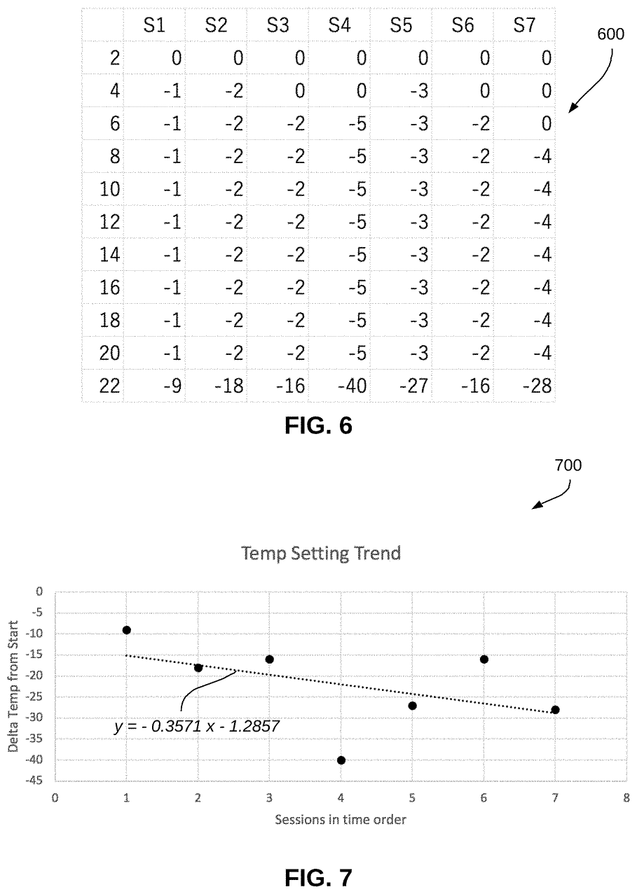

The raw data behind exemplary chart 500 of FIG. 5 are shown in a table 600 in FIG. 6. Session 1 is shown in column labeled “51,” Session 2 in “S2,” and so on. The numbers shown indicate the temperature difference from the initial temperature of 73° F. The left most column indicates the number of minutes elapsed from the start of the sessions. The final temperature, row 20, indicates the lowest temperature that the user perceived that was also tolerable at that particular time.

Referring now to FIG. 7, it is noted that the perception of cold and of tolerance is subjective and is not expected to be clearly uniform or monotonic. Plotting the final value of the data gives us what the patient perceives to be tolerable at that particular time. It is not consistent but drawing a trend line shows that the tolerable temperature is falling slowly over time.

This example shown in FIG. 7 indicates that the user is becoming more tolerant of cooler temperatures after repeated treatments. Assuming this trend continues, the patient should reach therapeutic temperatures of 59° F. in a total of about 36 sessions (using the formula shown in the plot) which is the goal.

While the trending analysis shown in FIG. 7 is a simple analysis that only provides a projection. Other algorithms may be produced to make on-the-fly decisions. For example, suppose the patient is manually setting the time of treatment to longer and longer times but isn't changing the temperature. An algorithm can be used to discover this and ask the patient if it would be acceptable to reduce the temperature for a limited time to see if the lower temperature can be tolerated. This type of algorithm can motivate the patient to more quickly reach therapeutic temperatures, thus urging the patient to achieve beneficial results. Such algorithms can also be used to limit the application of heat, cold or both for specific amounts of time and temperature, as instructed by a physician.

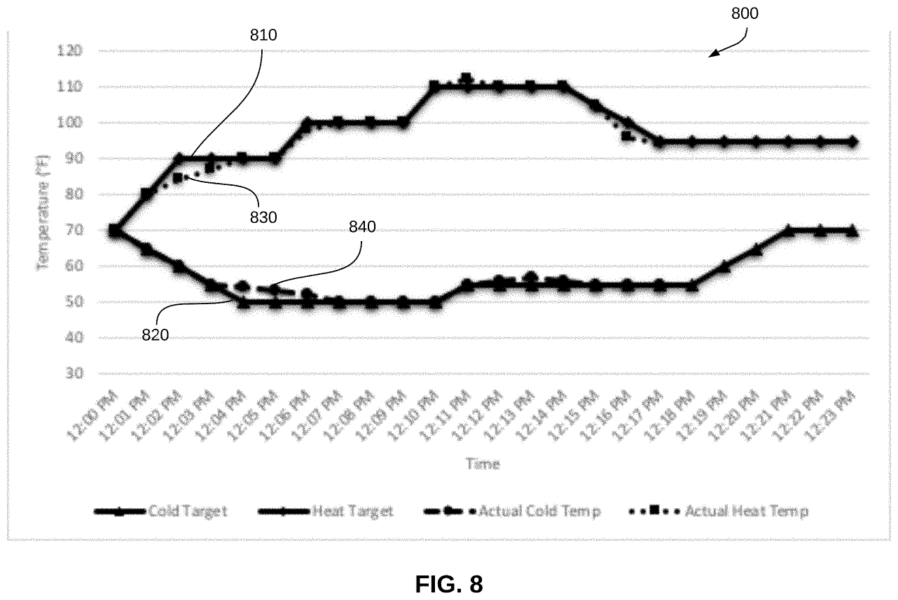

FIG. 8 is a graph showing typical treatment profiles for heating and cooling treatment protocols, illustrating the target temperatures and actual temperatures, as a function of time and implemented with an exemplary arthritis treatment system, such as system 300 of FIG. 3. Graph 800 includes a curve 810, showing the target temperature settings for the hot liquid reservoir, and a curve 820, showing the target temperature settings for the cold liquid reservoir. A curve 830 shows the actual temperature as measured in the hot liquid reservoir as the arthritis treatment system is activated. A curve 840 shows the actual temperature as measured in the cold liquid reservoir as the arthritis treatment system chills the cooling liquid according to the treatment protocol. In the embodiment shown, both the hot and cold sides of the arthritis treatment system are activated simultaneously, such that the patient is applying the heating pads to one part of his/her body, while the cooling pads are being applied to a different treatment area. Actual temperature measurements, as indicated by curves 830 and 840, stop at 12:18 pm, although the target temperature settings continue after that point in time.

As can be seen by curve 820 in FIG. 8, the temperature of the heating liquid starts at room temperature (70° F.) and is heated to 90° F. in 3 minutes. The system maintains that temperature for 3 minutes, and then increases the temperature of the heating liquid again to 100° F., and so on. At the same time, the cold side is being adjusted to lower temperatures at different rates.

The target temperature settings, as indicated by curves 810 and 820, can be programmed into the arthritis treatment system by a physician, a therapist, or the patient/user, or pre-programmed into the arthritis treatment system as optional treatment settings. If the profile is created by a physician or therapist and uploaded into the arthritis treatment system, then this particular profile can be named or numbered and placed in memory in the system controller, such that the profile cannot be modified by the patient. Such a profile can stay in the system controller indefinitely, or be removed or modified according to an authorized protocol, depending on the system settings.

If the arthritis treatment system is configured to allow patient control over the treatment protocol, the patient can, for example, be given the option of using one of several preset treatment protocols programmed into the system, or a fully- or partially-automated treatment setting. The controller can include, for example, a touchscreen that displays the measured temperatures of both hot and cold liquid reservoirs. In an exemplary usage mode, the user can adjust target temperatures in real time and watch the actual temperatures such that the user can manually adjust the temperatures as desired, thus effectively customizing the treatment experience. The patient can also be given the option of programming a customized treatment protocol. In either usage scenario, a programmed treatment protocol can be used repeatedly over the course of several sessions. Additionally, the patient can be provided with the option of manually adjusting the treatment protocol settings, either prior to treatment initialization or during the treatment session, thus providing the patient with customizable control over his/her treatment experience.

As an example, an arthritis patient can place the cold pads on his/her knees and hot pads on his/her hands in one treatment session, while at another session he/she can choose to put the cold pads on his/her ankles and the hot pads on his/her elbows. Each of these treatment options can be programmed into the system controller for repeatable use. The controller in the arthritis treatment system can include preset limitations for, for example, temperature thresholds or timing. For instance, the temperature can be limited to a high setting between 130-140° F., while the cold temperatures will be limited to not less than 32° F. The treatment session time can also be limited to a preset or physician-prescribed setting.

With appropriate permissions from the patients and prescribing physicians, the use data for each patient that uses the device can be selectively collected for further research. For instance, while hot and cold treatment systems have been in use for many years, there are limited studies relating the benefit of particular therapy times and temperatures to specific ailments. There are even some white papers and online sites that claim that cold packs are contraindicated for arthritis and indeed 32° F. cold packs should be contraindicated for certain patients but 55° F. should not. Collection of use data for the present system can provide valuable information for research to further treatment protocols for arthritis and other ailments.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention.

Accordingly, many different embodiments stem from the above description and the drawings. It will be understood that it would be unduly repetitious and obfuscating to literally describe and illustrate every combination and subcombination of these embodiments. As such, the present specification, including the drawings, shall be construed to constitute a complete written description of all combinations and subcombinations of the embodiments described herein, and of the manner and process of making and using them, and shall support claims to any such combination or subcombination.

For instance, verbal instructions and questions can be built into the device to help the user setup an account, setup the machine, choose between heat and cold, and even assist in entering new automated therapy steps of time and temperature. Many questions can be asked by the device and many can be answered by the user with simple “yes”/“no” answers, thus assisting mobility impaired patients that may not be able to tolerate extensive touch inputs or complicated setup procedures. Furthermore, specific setup and feedback questions can be asked verbally by the device then recording the response. This response can then be saved as historical information. For example, the question, “Have you read and accepted the HIPAA notification?” can be asked by the device via a recorded voice, and the answer can be recorded and stored, such that the patient can be urged to read it, if they haven't yet read the specific wording and even display it on a display for the patient to review. Voice instructions, controller to user, can be recorded instructions or generated vocalization. Voice instructions and answers, user to controller, can be sensed by the controller with any microphone, where microphone can be any microphone such as a speaker used in a reciprocal fashion or a simple electret, such as those produced by Panasonic and many others.

As another exemplary variation, thermistors and thermocouples can be used to sense temperature within the system, such as in the fluid going to or from the pad. In an embodiment, the controller monitors this temperature and can be programmed to act based on this information. The sensor can be, for example, placed in the fluid or in close proximity coupled to the fluid through a thermally conductive material. A thermistor or thermocouple can also sense the working environment temperature of the device. By monitoring the environmental conditions around the system, variables such as the current temperature and humidity, including the air, can be used to in the algorithms to adjust the cooling and heating pad temperatures.

Furthermore, pressure sensors can be used to detect normal operation, fluid levels, leaks, and/or blockages. Flow sensors can also be used to detect normal operation, leaks and blockages. Level sensors can be used to sense the level of the fluid in each reservoir. The controller can be programmed to act in a way appropriate to the sensed condition.

Pumps can be powered by or controlled by alternating current (AC), direct current (DC), pulse width modulation (PWM), digital, or other mechanisms. For example, the controller can produce PWM signals that vary the voltage with the changing needs of the flow rate required.

Suitable heating devices include, but are not limited to, resistive heaters, cartridge heaters, tubular heaters, infrared heaters, and induction heaters.

Controllers can be microcontroller, computer, digital logic including field programmable gate arrays (FPGAs) and programmable logic devices (PLDs), analog circuitry, or other mechanisms.

The user interface can include a display including a touch sensitive screen, a keypad, mouse or trackpad, or a specialized button, multiple buttons, knobs, and/or sliders.

The cooling and heating sides of the arthritis treatment system can also be used independently. Additionally, the arthritis treatment system can be integrated with other common forms of therapy, such as transcutaneous electrical nerve stimulation (TENS) units, compression devices, topical lotions, and pharmaceuticals. The Peltier component could be replaced with a common refrigeration unit or a vacuum cold producing method.

While the arthritis treatment system described herein, is optimized for use by arthritis patients, it may also be used for treatment of other ailments. The Peltier cooling surface should be colder than the desired temperature at the pad due to fluid heating by thermal conduction through the reservoirs and tubing, and such temperature differences throughout the system can be monitored using sensors at strategic locations. The tubing can be, for example, approximately 12 feet long, and the specific length can be adjusted for user convenience. The tubing can optionally be covered, in whole or in part, with a suitable insulation material, such as aerogel or foam (e.g., ARMAFLEX® insulation). The system can also provide temperature settings that are lower or higher than those expected for arthritis treatment. Accordingly, the cooling and heating fluid mixtures can be adjusted to support, for example, below freezing temperatures, such as by mixing deionized water and glycol or another suitable fluid.

In the specification, there have been disclosed embodiments of the invention and, although specific terms are employed, they are used in a generic and descriptive sense only and not for purposes of limitation. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

What is claimed is:

1. A heating and cooling platform comprising:
a hot fluid chamber in thermal communication with a heating device;
a cold fluid chamber in thermal communication with a cooling device;
a first conduit in fluid communication with the hot fluid chamber and a second conduit in fluid communication with the cold fluid chamber, the first conduit and the second conduit each comprising at least one connector for joining an interface;
at least one pump for moving fluid through the first conduit and the second conduit; and
a controller comprising a processor configured to provide instructions to the heating device, the cooling device, and the at least one pump.

2. The heating and cooling platform of claim 1, wherein the interface is a treatment pad.

3. The heating and cooling platform of claim 1, wherein the interface is a plurality of treatment pads.

4. The heating and cooling platform of claim 1 further comprising a heat exchanger in thermal communication with the first conduit and the second conduit.

5. The heating and cooling platform of claim 4 further comprising a second pump for circulating fluid through the heat exchanger.

6. The heating and cooling platform of claim 1, wherein the hot fluid chamber and the cold fluid chamber are maintained at distinct temperatures.

7. The heating and cooling platform of claim 1 further comprising a user interface in operable communication with the controller.

8. The heating and cooling platform of claim 1 further comprising a memory for storing patient information, usage data and/or treatment session settings.

9. The heating and cooling platform of claim 8, wherein the patient information includes at least one of patient weight, patient height, patient age, body temperature, patient sex, and diagnosed ailment.

10. The heating and cooling platform of claim 1, wherein the interface comprises a vital sign sensor selected from the group consisting of a body temperature sensor, a pulse rate sensor, and an oxygen saturation sensor.

11. The heating and cooling platform of claim 1 further comprising at least one device sensor located within the hot fluid chamber or the cold fluid chamber for sensing at least one of temperature, pressure, and a fluid level within the chamber.

12. The heating and cooling platform of claim 1 further comprising a first flow sensor in the first conduit and a second flow sensor in the second conduit, each of the first flow sensor and the second flow sensor in operable communication with the controller.

13. The heating and cooling platform of claim 1, wherein the at least one connector is a plurality of connectors for joining a plurality of interfaces.

14. The heating and cooling platform of claim 13, wherein the plurality of interfaces is a plurality of treatment pads.
